(19) United States
(12) Patent Application Publication
(10) Pub. No.: US 2015/0342964 A1
(43) Pub. Date: Dec. 3, 2015

(54) DEXAMETHASONE POLYMER IMPLANT FOR PAIN

(71) Applicant: Warsaw Orthopedic, Inc., Warsaw, IN (US)

(72) Inventors: Jonathan Gray, Collierville, TN (US); Jeffrey C. Marx, Germantown, TN (US); William F. McKay, Memphis, TN (US); Josee Roy, Memphis, TN (US); Jared T. Wilsey, Memphis, TN (US)

(73) Assignee: Warsaw Orthopedic, Inc., Warsaw, IN (US)

(21) Appl. No.: 14/291,131

(22) Filed: May 30, 2014

(51) Int. Cl. A61K 31/573; A61K 45/06; A61K 31/4168; A61K 47/34

(52) U.S. Cl. CPC A61K 31/573; A61K 47/34; A61K 45/06; A61K 31/4168

(57) ABSTRACT

A biodegradable drug implant for reducing, preventing or treating pain and/or inflammation in a patient is provided. The biodegradable drug implant comprises dexamethasone and a polylactic acid polyglycolic acid (PLGA) copolymer matrix, wherein the dexamethasone comprises from about 50% to about 80% by weight of the drug implant. Methods of treating pain in a patient are also provided.

### BACKGROUND

Radicular pain is pain that arises from an irritation or inflammation of spinal nerves or roots at the connection to the spinal column. This results in symptoms that “radiate” from one site to another. The pain often originates at or near the vertebral column and travels down one or both extremities. A common form of radicular pain is lumbar radicular pain, also known as “sciatica,” which radiates along the sciatic nerve from the lower spine to the lower back, gluteal muscles, back of the upper thigh, calf and foot. Lumbar radicular pain is a sharp shooting pain and is usually associated with neurologic signs or symptoms. Radiculopathic pain is, at least in part, a result of inflammatory factors such as cytokinins in the vicinity of the spinal nerve root compression.

Based on the inflammatory etiology of radicular pain, analgesics and epidural steroid injections are a conventional intervention with therapeutic value in approximately half of patients. These measures may alleviate pain enough to allow more physical activity. Epidural steroid injections involve injecting a steroid, either alone or together with an analgesic, local anesthetic (lidocaine or bupivacaine), and/or or saline directly into the problem area in the low back to treat the inflammation that is irritating the nerve root. Dexamethasone, triamcinolone acetate, and methylprednisolone acetate are commonly used steroids. Epidural steroid injections are short acting and must be repeated at regular intervals, requiring fluoroscopy or x-ray to ensure accurate placement, and necessitating a physician visit for each injection. Currently, these injections are not FDA approved for radicular pain.

In a portion of patients, oral or injectable medical treatment with steroids or strong analgesics such as opioids are necessary. Opioids are addictive, have an abuse potential, as such they are controlled substances, and prescriptions must be reported to prescription monitoring programs in most states. They must only be used for a limited period (e.g., 2-4 weeks). Because of the abuse potential, long acting opioids such as fentanyl patch are restricted by the Food and Drug Administration under a REMS program. Side effects include sedation and an inability to operate machinery or drive. Their regular use may cause nausea and constipation. Additionally, high doses are often required. For these reasons, clinicians have been moving away from prescribing these agents.

In other patients who achieve limited success with these therapeutic modalities, especially patients with unresolved radicular pain, surgical treatment is necessary. Surgery is indicated for those patients with progressive neurological deficits or severe lumbar radicular pain that is unresponsive to minimally invasive interventional pain relief measures. Surgery is considered for only a select group of patients since it is a permanent procedure which may be unsuccessful or lead to hemiplegia or disability.

Drug delivery pumps have been used in radicular pain to deliver pain medication directly to the fluid around the spinal cord. Delivery of medication directly to the pain receptors near the spine instead of going through the circulatory system may result in more effective pain relief at smaller doses and less side effects than would be required via oral administration. However, these pumps require an intrathecal catheter to deliver the medication to the area where fluid flows around the spinal cord. Possible complications include the catheter or drug pump moving within the body or wearing through the skin. The catheter could leak, tear, kink, or become disconnected. The pump could stop because the battery has run out or because of failure of another part of the infusion system. Inflammatory masses may occur at the tip of the catheter which in some cases may lead to paralysis.

There is a need, therefore, to develop a therapeutic system for the long term treatment of pain (e.g., radicular pain) with minimal side effects which could deliver an effective agent such as an anti-inflammatory agent or analgesic pain relieving agent directly or very near the source of pain generation (e.g., the spinal area).

Another disease that needs treatment is obesity. More particularly, obesity is an important and growing public health problem around the world and is associated with premature mortality, chronic morbidity, and increased health care use. In the United States, approximately, one third of adults are obese. Pharmacotherapeutic agents to induce weight loss may reduce appetite or increase satiety, reduce nutrient absorption, or increase energy expenditure. Despite the plethora of products available either by prescription or over-the-counter for weight loss, weight loss with therapeutic agents is generally modest compared to placebo. Obesity remains common. Therapeutics for treating obesity include anorectic agents, stimulants such as amphetamines, gastrointestinal lipase inhibitors, endocannabinoid receptor antagonists, and appetite suppressants. Amphetamines are severely restricted due to their abuse potential. Amongst the medications marketed for weight loss, there have been several instances of market withdrawal due to serious side effects. Extended-release oral products have been developed to provide a controlled-release of such medications. However, weight loss is often disappointing, recidivism is high with current drug treatments, current medications for weight loss are costly, and each has associated side effects. Discovery of successful alternative non-stimulant weight loss agents and routes of administration are needed. Additionally, there is a need for novel controlled release drug delivery systems to deliver these medications to target tissues.

One pharmaceutical that is known to the medical profession is dexamethasone. Dexamethasone, which is a corticosteroid, is also known as (8S, 9R, 10S, 11S, 13S, 14S, 16R, 17R)-9-Fluoro-11,17-dihydroxy-17-(2-hydroxyacetyl)-10,13,16-trimethyl-6,7,8,11,12,14,15,16-octahydro-cyclopenta[a]phenanthren-3-one, and is available from various pharmaceutical manufacturers. Two known commercially available forms are dexamethasone acetate and dexamethasone sodium phosphate.

Dexamethasone is known for a number of uses, including treatment of inflammatory and autoimmune diseases such as rheumatoid arthritis. However, to date, dexamethasone has not been optimized as an effective treatment for acute or chronic pain or radicular pain. Dexamethasone also has not been recognized for the treatment of obesity. Thus, there is a need to develop effective formulations and delivery systems containing dexamethasone for these applications.

### SUMMARY

Compositions and methods are provided comprising dexamethasone or its pharmaceutically acceptable salts that are administered in order to reduce, prevent or treat radicular pain and/or inflammation, or induce weight loss.

According to one embodiment, there is a pharmaceutical formulation comprising: dexamethasone, wherein the dexamethasone comprises from about 50% to about 80% by weight of the formulation, and a polylactic acid polyglycolic acid (PLGA) copolymer matrix. The pharmaceutical composition may for example, be a biodegradable drug implant for reducing, preventing or treating radicular pain and/or inflammation in a patient in need of such treatment. The drug implant may: (i) consist of only the dexamethasone (or one or more of its pharmaceutically acceptable salts) and the biodegradable PLGA copolymer matrix; or (ii) consist essentially of the dexamethasone (or one or more of its pharmaceutically acceptable salts) and the biodegradable PLGA copolymer matrix; or (iii) comprise the dexamethasone (or one or more of its pharmaceutically acceptable salts) and the biodegradable PLGA copolymer matrix and one or more other active ingredients, surfactants, excipients or other ingredients or combinations thereof. When there are other active ingredients, surfactants, excipients or other ingredients or combinations thereof in the formulation, in some embodiments these other compounds or combinations thereof comprise less than 30 wt. %, less than 25 wt. %, less than 20 wt. %, less than 19 wt. %, less than 18 wt. %, less than 17 wt. %, less than 16 wt. %, less than 15 wt. %, less than 14 wt. %, less than 13 wt. %, less than 12 wt. %, less than 11 wt. %, less than 10 wt. %, less than 9 wt. %, less than 8 wt. %, less than 7 wt. %, less than 6 wt. %, less than 5 wt. %, less than 4 wt. %, less than 3 wt. %, less than 2 wt. %, less than 1 wt. % or less than 0.5 wt. % of the drug implant.

According to another embodiment, there is a biodegradable drug implant for reducing, preventing or treating radicular pain and/or inflammation in a patient in need of such treatment, the biodegradable drug implant comprising: dexamethasone and a polylactic acid polyglycolic acid (PLGA) copolymer matrix, wherein the dexamethasone comprises from about 50% to about 80% by weight of the drug implant.

In some embodiments, the drug implant is configured for placement under the skin and releases the dexamethasone over a period of at least about 30 days. In some embodiments, the drug implant releases the dexamethasone in an amount of about 0.1 mg to 4 mg over a period of 30 to 60 days after placement beneath the skin.

In some embodiments, the drug implant releases the dexamethasone in an amount of about 0.35 mg over a period of 30 to 60 days after placement beneath the skin. In various embodiments, the drug implant releases the dexamethasone in an amount of about 0.7 mg over a period of 30 to 60 days after placement beneath the skin. In some embodiments, the polylactic acid polyglycolic acid (PLGA) copolymer matrix comprises at least 40% of the weight of the drug implant.

In various embodiments, the dexamethasone comprises a burst release surface that releases a bolus dose of the dexamethasone that is 5% to 15% of the dexamethasone loaded in the drug implant within 24 hours. In some embodiments, the implant is placed at or near the spine.

In various embodiments, the drug implant further comprises a clonidine compound, an analgesic, a GABA compound, a corticosteroid, a muscle relaxant, a non-steroidal anti-inflammatory compound, an anesthetic or a combination thereof. In some embodiments, the drug implant comprises clonidine. In various embodiments, the implant releases dexamethasone within 7 days after the implant is implanted under the skin.

According to another embodiment, there is kit comprising: a biodegradable drug implant comprising dexamethasone and a polylactic acid polyglycolic acid copolymer matrix, wherein the drug implant is designed to be implanted in or near the spine; and instructions for use. In some embodiments, the kit is used for treating radicular pain.

According to another embodiment, there is a method for reducing, preventing or treating radicular pain in a patient in need of such treatment, the method comprising administering a biodegradable drug implant to a target tissue site beneath the skin of the patient, the biodegradable drug implant comprising dexamethasone in an amount from about 50% to 80% of the weight of the drug implant, and a polylactic acid polyglycolic acid (PLGA) copolymer matrix, wherein the drug implant releases the dexamethasone over a period of at least about 30 days. In some embodiments, the radicular pain is caused by sciatica. In various embodiments, the biodegradable implant is administered at or near the spine.

According to another embodiment, there is a biodegradable drug implant for treating weight loss in a patient in need of such treatment, the biodegradable drug implant comprising dexamethasone and a biodegradable polymer matrix, wherein the dexamethasone comprises from about 50% to about 80% by weight of the drug implant. In some embodiments, the biodegradable polymer matrix comprises a polylactic acid polyglycolic acid (PLGA) copolymer matrix.

According to another embodiment, there is a method for weight loss in a patient in need of such treatment, the method comprising administering a biodegradable drug implant to a target tissue site beneath the skin of the patient, the biodegradable drug implant comprising dexamethasone in an amount from about 50% to 80% of the weight of the drug implant, and a biodegradable polymer matrix, wherein the drug implant releases the dexamethasone over a period of at least about 30 days.

In various embodiments, the biodegradable polymer matrix comprises a polylactic acid polyglycolic acid (PLGA) copolymer matrix. In some embodiments, the weight loss occurs within one week after the implant is implanted beneath the skin and over a period of at least 3 weeks. In some embodiments, the weight loss occurs within one week after the implant is implanted beneath the skin and over a period of at least 30 days. In some embodiments, the biodegradable drug implant is administered at or near the spine. In various embodiments, the drug implant further comprises an anorectic, a corticosteroid, an anti-inflammatory agent, a stimulant or a combination thereof.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a schematic dorsal view of the spine and sites within a patient where a dexamethasone implant of the present application can be implanted beneath the skin.

FIG. 2 is a graphic illustration of the thermal paw withdrawal latency as a percent of pre-injury baseline employing a biodegradable dexamethasone implant of the present application.

FIG. 3 is a graphic illustration of the von Frey monofilament test for mechanical allodynia employing a biodegradable dexamethasone implant of the present application.

It is to be understood that the figures are not drawn to scale. Further, the relation between objects in a figure may not be to scale, and may, in fact, have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

### DEFINITIONS

It is noted that, as used in this specification and the appended claims, the singular forms “a,” “an,” and “the,” include plural referents unless expressly and unequivocally limited to one referent. Thus, for example, reference to “a drug implant” includes one, two, three or more matrices.

An implantable medical device includes that the device can be implanted into the human body. In various embodiments, the medical device is implanted into the back. The medical device allows release of the drug or therapeutic agent. A medical device includes a drug depot, which can be solid, semi-solid or in gel form.

An “implant” and “implantable medical device” are used interchangeably herein, and include any biodegradable device for implantation beneath the skin which is capable of delivering a therapeutic level of a drug to the site of pain (e.g., radicular pain).

To “implant” to “place” and to “insert” are equivalent as used herein and mean to place an object in the desired site by any means capable of placing the object at that site.

The term “implantable” as utilized herein refers to a biocompatible depot (e.g., device) retaining potential for successful placement within a mammal. The expression “implantable depot” and expressions of the like utilized herein, refer to an object implantable through surgery, injection, or other suitable means whose primary function is achieved either through its physical presence or mechanical properties.

A “drug implant” is a composition in which the dexamethasone is administered to the body. Thus, a drug implant may comprise a physical structure (e.g., strip, pellet) to facilitate implantation and retention in a desired site (e.g., a disc space, a spinal canal, a tissue of the patient, particularly at or near a site of chronic pain, etc.). The drug implant (e.g., pellet) may also comprise the drug itself. The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API”. It will be understood that unless otherwise specified a “drug” formulation may include more than one therapeutic agent, wherein exemplary combinations of therapeutic agents include a combination of two or more drugs. The drug provides a concentration gradient of the therapeutic agent for delivery to the site. In various embodiments, the drug implant (e.g., pellet) provides an optimal drug concentration gradient of the therapeutic agent at a distance of up to about 0.01 cm to about 20 cm from the administration site and comprises dexamethasone.

A “therapeutically effective amount” or “effective amount” is such that when administered, the drug results in alteration of the biological activity, such as, for example, inhibition of inflammation, reduction or alleviation of pain or spasticity, improvement in the condition through muscle relaxation, etc. The dosage administered to a patient can be a single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments, the formulation is designed for immediate release. In other embodiments, the formulation is designed for sustained release. In other embodiments, the formulation comprises one or more immediate release surfaces and one or more sustained release surfaces.

An “implant” includes but is not limited to capsules, microspheres, microparticles, microcapsules, microfibers particles, nanospheres, nanoparticles, coating, layers, matrices, wafers, pills, pellets, emulsions, liposomes, micelles, gels, or other pharmaceutical delivery compositions or a combination thereof. Suitable materials for the implants (e.g., pellet) are pharmaceutically acceptable biodegradable and/or any bioabsorbable materials that are preferably FDA approved or GRAS materials. These materials can be polymeric or non-polymeric, as well as synthetic or naturally occurring, or a combination thereof.

The “pellet” of the present application provides a 3-D fiber of interconnecting pores, which acts as a pliant scaffold for cell migration and/or drug release.

The “PLGA copolymer matrix” of the present application provides synthetic polymers having lactic acid or glycolic acid ester linkages subject to hydrolytic degradation to non-toxic tissue compatible absorbable components, including polylactic acid or polyglycolic acid. The matrix may be monofilamentary or braided, absorbable or non-absorbable.

The term “biodegradable” includes that all or parts of the drug depot (e.g., pellet) will degrade over time by the action of enzymes, by hydrolytic action and/or by other similar mechanisms in the human body. In various embodiments, “biodegradable” includes that the implant (e.g., pellet) can break down or degrade within the body to non-toxic components after or while a therapeutic agent has been or is being released. By “bioerodible” it is meant that the implant will erode or degrade over time due, at least in part, to contact with substances found in the surrounding tissue, fluids or by cellular action. By “bioabsorbable” it is meant that the implant (e.g., pellet) will be broken down and absorbed within the human body, for example, by a cell or tissue. “Biocompatible” means that the implant (e.g., pellet) will not cause substantial tissue irritation or necrosis at the target tissue site.

In some embodiments, the drug implant (e.g., pellet) has pores that allow release of the drug from the implant (e.g., pellet). The drug implant (e.g., pellet) will allow fluid in the implant (e.g., pellet) to displace the drug. However, cell infiltration into the implant (e.g., pellet) will be prevented by the size of the pores of the implant (e.g., pellet). In this way, in some embodiments, the implant (e.g., pellet) should not function as a tissue scaffold and allow tissue growth. Rather, the drug implant (e.g., pellet) will solely be utilized for drug delivery. In some embodiments, the pores in the drug implant (e.g., pellet) will be less than 250 to 500 microns. This pore size will prevent cells from infiltrating the drug implant (e.g., pellet) and laying down scaffolding cells. Thus, in one embodiment, the drug will elute from the drug implant (e.g., pellet) as fluid enters the drug implant, but cells will be prevented from entering. In some embodiments, where there are little or no pores, the drug will elute out from the drug implant (e.g., pellet) by the action of enzymes, by hydrolytic action and/or by other similar mechanisms in the human body. In some embodiments, the drug implant (e.g., pellet) will function to allow influx of cells and tissue and it will function as a scaffold.

The phrase “sustained release” or “sustain release” (also referred to as extended release or controlled release) is used herein to refer to one or more therapeutic agent(s) that is introduced into the body of a human or other mammal and continuously or continually releases a stream of one or more therapeutic agents over a predetermined or programmed time period and at a therapeutic level sufficient to achieve a desired therapeutic effect throughout the predetermined time period. Reference to a continuous or continual release stream is intended to encompass release that occurs as the result of biodegradation in vivo of the drug implant, or a pellet or component thereof, or as the result of metabolic transformation or dissolution of the therapeutic agent(s) or conjugates of therapeutic agent(s).

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug.

In some embodiments, sustained release and immediate release formulations may be used in conjunction. The sustained release and immediate release may be in one or more of the same pellet. In various embodiments, the sustained release and immediate release may be part of separate drug implants. For example a bolus or immediate release formulation of dexamethasone may be placed at or near the target site and a sustained release formulation may also be placed at or near the same site. Thus, even after the bolus becomes completely accessible, the sustained release formulation would continue to provide the active ingredient for the intended tissue.

In various embodiments, the drug implant can be designed to cause an initial burst dose of a therapeutic agent within the first twenty-four to seventy-two hours after implantation. “Initial burst” or “burst effect,” “burst release” or “bolus dose” refer to the release of therapeutic agent from the implant (e.g., fiber, pellet, strip, etc.) during the first twenty-four hours to seventy-two hours after the depot (e.g., fiber) comes in contact with an aqueous fluid (e.g., interstitial fluid, synovial fluid, cerebral spinal fluid, etc.). The “burst effect” is believed to be due to the increased release of therapeutic agent from the drug implant. In some embodiments, the drug implant has one or more burst release surfaces that releases about 10%, 15%, 20%, 25%, 30%, 35%, 45%, to about 50% of the drug over 24 to 48 hours.

In alternative embodiments, the drug implant is designed to avoid or reduce this initial burst effect (e.g., by applying an outer polymer coating to the drug implant).

“Treating” or “treatment” of a disease or condition refers to executing a protocol that may include administering one or more drugs to a patient (human, other normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition. In addition, treating or treatment does not require complete alleviation of signs or symptoms, does not require a cure, and specifically includes protocols that have only a marginal effect on the patient. “Reducing pain and/or inflammation” includes a decrease in pain and/or inflammation and does not require complete alleviation of pain and/or inflammation signs or symptoms, and does not require a cure. In various embodiments, reducing pain and/or inflammation includes even a marginal decrease in pain and/or inflammation. By way of example, the administration of the effective dosage of dexamethasone may be used to prevent, treat or relieve the symptoms of pain and/or inflammation for various diseases or conditions causing radicular pain. These diseases or conditions include, but are not limited to, spinal disc herniation (e.g., sciatica), pinched nerve, lower back pain, cancer, tissue pain and pain associated with injury or repair of cervical, thoracic, and/or lumbar vertebrae or intervertebral discs, articular joint, muscles, spondilothesis, stenosis, discogenic back pain or the like.

One chronic condition is sciatica. In general, sciatica is an example of pain that can transition from acute to neuropathic pain. Sciatica refers to pain associated with the sciatic nerve which runs from the lower part of the spinal cord (the lumbar region), down the back of the leg and to the foot. Sciatica generally begins with a herniated disc. The herniated disc itself leads to local immune system activation. The herniated disc also may damage the nerve root by pinching or compressing it, leading to additional immune system activation in the area. In various embodiments, the dexamethasone may be used to reduce, treat, or prevent sciatic pain and/or inflammation by locally administering the dexamethasone at one or more target tissue sites (e.g., nerve root, dorsal root ganglion, focal sites of pain, at or near the spinal column, etc.).

In some embodiments, the drug implant can be used to treat one or more target tissue sites that are involved in conditions/diseases, such as for example, rheumatoid arthritis, osteoarthritis, sciatica, lower back pain, lower extremity pain, upper extremity pain, cancer, tissue pain and pain associated with injury or repair of cervical, thoracic, and/or lumbar vertebrae or intervertebral discs, rotator cuff, articular joint, tendons, ligaments, muscles, a surgical wound site or an incision site, postoperative pain or the like.

The term “implantable” as utilized herein refers to a biocompatible device (e.g., pellet) retaining potential for successful placement within a mammal. The expression “implantable device” and expressions of the like refers to an object implantable through surgery, injection, or other suitable means whose primary function is achieved either through its physical presence or mechanical properties.

“Localized” delivery includes delivery where one or more drugs are deposited within a tissue, for example, a nerve root of the nervous system or a region of the brain, or in close proximity (within about 0.1 cm, or within about 10 cm, for example) thereto. For example, the drug dose delivered locally from the drug implant may be, for example, 5%, 10%, 15%, 20%, 25%, 30%, 35%, 40%, 45%, 50%, 55%, 60%, 65%, 70%, 75%, 80%, 85%, 90%, 95%, 99%, or 99.9% less than the oral dosage or injectable dose. In turn, systemic side effects, such as, for example, gastrointestinal ulceration, adrenal suppression, etc. may be reduced or eliminated.

The term “mammal” refers to organisms from the taxonomy class “mammalian,” including but not limited to humans, other primates such as chimpanzees, apes, orangutans and monkeys, rats, mice, cats, dogs, cows, horses, etc.

The phrase “pain management medication” includes one or more therapeutic agents that are administered to prevent, alleviate or remove pain entirely. These include anti-inflammatory agents, muscle relaxants, analgesics, anesthetics, narcotics, and so forth, and combinations thereof.

The phrase “release rate profile” refers to the percentage of active ingredient that is released over fixed units of time, e.g., mcg/hr, mcg/day, 10% per day for ten days, etc. As persons of ordinary skill know, a release rate profile may, but need not, be linear. By way of a non-limiting example, the drug implant may be a matrix that releases the dexamethasone over a period of time.

The term “solid” is intended to mean a rigid material, while, “semi-solid” is intended to mean a material that has some degree of flexibility, thereby allowing the implant (e.g., pellet, fiber, strip) to bend and conform to the surrounding tissue requirements.

“Targeted delivery system” provides delivery of one or more drugs implants (e.g., pellet, fibers, strip, etc.) at or near the target site as needed for treatment of pain, inflammation or other disease or condition.

The abbreviation “PLGA” refers to polylactide-co-glycolide).

The abbreviation “PLA” refers to polylactide.

Reference will now be made in detail to certain embodiments of the application, examples of which are illustrated in the accompanying figures. While the application will be described in conjunction with the illustrated embodiments, it will be understood that they are not intended to limit the application to those embodiments. On the contrary, the application is intended to cover all alternatives, modifications, and equivalents that may be included within the application as defined by the appended claims.

### Pain and Dexamethasone

Compositions and methods are provided comprising dexamethasone or its pharmaceutically acceptable salts that are administered in order to reduce, prevent or treat radicular pain and/or inflammation, or induce weight loss.

There are many structures in the back that can cause severe pain. These include muscles, ligaments, tendons, bones, joints and discs. The outer rim of the disc can be a source of significant back pain due to its rich nerve supply and tendency towards injury. Back pain can be divided into three types: axial pain, referred pain and radicular pain. Radicular pain is deep and constant, often following the nerve down the leg and accompanied by numbness of tingling and muscle weakness. The most common example of this type of pain is sciatic pain that radiates along the sciatic nerve, down the back of the thigh and calf into the foot. The pain is the result of injury to the spinal nerve from causes such as disc protrusion or bulge, arthritic changes or a narrowing of the opening through which the nerve exits.

One or more drug implants may be used to treat conditions of radicular pain and/or inflammation in chronic conditions including spinal disc herniation (e.g., sciatica), pinched nerve, lower back pain, cancer, tissue pain and pain associated with injury or repair of cervical, thoracic, and/or lumbar vertebrae or intervertebral discs, articular joint, muscles, spondolithesis, stenosis, discogenic back pain or the like.

Inflammatory chemicals (e.g., Substance P, PLA2, arachadonic acid, TNF-α, IL-1, and prostaglandin E2) and immunologic mediators can generate radicular pain. Since the vast majority of the pain stems from chemical inflammation, corticosteroids such as dexamethasone when administered directly (or very near) the source of pain generation, alleviate the inflammatory response caused by chemical and mechanical sources of pain. Steroids, such as dexamethasone, also work by reducing the activity of the immune system to react to inflammation associated with nerve or tissue damage. Dexamethasone, at the site of pain and inflammation, controls local inflammation while “flushing” out inflammatory proteins and chemicals from the local area that may contribute to and exacerbate pain.

According to one embodiment, there is a pharmaceutical formulation comprising: dexamethasone, wherein the dexamethasone comprises from about 50% to about 80% by weight of the formulation, and a polylactic acid polyglycolic acid (PLGA) copolymer matrix. The pharmaceutical composition may for example, be a biodegradable drug implant for reducing, preventing or treating radicular pain and/or inflammation in a patient in need of such treatment. The drug implant may: (i) consist of only the dexamethasone (or one or more of its pharmaceutically acceptable salts) and the biodegradable PLGA copolymer matrix; or (ii) consist essentially of the dexamethasone (or one or more of its pharmaceutically acceptable salts) and the biodegradable PLGA copolymer matrix; or (iii) comprise the dexamethasone (or one or more of its pharmaceutically acceptable salts) and the biodegradable PLGA copolymer matrix and one or more other active ingredients, surfactants, excipients or other ingredients or combinations thereof. When there are other active ingredients, surfactants, excipients or other ingredients or combinations thereof in the formulation, in some embodiments these other compounds or combinations thereof comprise less than 30 wt. %, less than 25 wt. %, less than 20 wt. %, less than 19 wt. %, less than 18 wt. %, less than 17 wt. %, less than 16 wt. %, less than 15 wt. %, less than 14 wt. %, less than 13 wt. %, less than 12 wt. %, less than 11 wt. %, less than 10 wt. %, less than 9 wt. %, less than 8 wt. %, less than 7 wt. %, less than 6 wt. %, less than 5 wt. %, less than 4 wt. %, less than 3 wt. %, less than 2 wt. %, less than 1 wt. % or less than 0.5 wt. % of the drug depot.

In some embodiments, the dexamethasone is from about 50% to about 80% by weight of the implant. In some embodiments, the dexamethasone is from about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% to about 80% by weight of the implant. In some embodiments, the implant is monolithic, e.g. having the dexamethasone homogeneously distributed through the copolymer matrix. As such, no detrimental fluctuations in rate of dexamethasone release occur because of uneven distribution of dexamethasone in the copolymer matrix. In some embodiments, the polymeric matrix will not be fully degraded until the drug load has been released. In some embodiments, the polylactic acid polyglycolic acid (PLGA) copolymer matrix comprises at least about 20 weight percent of the implant.

In some embodiments, the drug implant is configured for placement under the skin and releases the dexamethasone over a period of at least about 30 days. In some embodiments, the drug implant releases the dexamethasone in an amount of about 0.1 mg to 4 mg over a period of 30 to 60 days after placement beneath the skin. In various embodiments, the drug implant releases the dexamethasone in an amount of about 0.35 mg over a period of 30 to 60 days after placement beneath the skin. In some embodiments, the drug implant releases the dexamethasone in an amount of about 0.7 mg over a period of 30 to 60 days after placement beneath the skin. In various embodiments, the implant is placed at or near the spine. In various embodiments, the implant releases dexamethasone within 7 days after the implant is implanted under the skin.

In various embodiments, dexamethasone may be released from the biodegradable drug implant at a dose of about 10 micrograms to about 80 mg/day, about 2.4 ng/day to about 50 mg/day, about 50 ng/day to about 2.5 mg/day, about 250 ng/day to about 250 micrograms/day, about 250 ng/day to about 50 micrograms/day, about 250 ng/day to about 25 micrograms/day, about 250 ng/day to about 1 micrograms/day, about 300 ng/day to about 750 ng/day or about 0.50 micrograms/day. In various embodiments, the dose may be about 0.01 to about 10 mg/day or about 1 ng to about 120 mg/day. In one exemplary embodiment, the dexamethasone is dexamethasone sodium phosphate or a dexamethasone acetate.

According to another embodiment, there is a biodegradable drug implant comprising: dexamethasone in an amount from about 50% to about 80% by weight of the drug implant, and a polylactic acid polyglycolic acid (PLGA) copolymer matrix comprises at least 60% of the weight of the drug implant.

According to another embodiment, there is a biodegradable drug implant comprising: dexamethasone, wherein the dexamethasone comprises a burst release surface that releases a bolus dose of the dexamethasone that is 5% to 15% of the dexamethasone loaded in the drug implant within 24 hours.

In some embodiments, the drug implant comprises a clonidine compound, an analgesic, a GABA compound, a corticosteroid, a muscle relaxant, a non-steroidal anti-inflammatory compound, an anesthetic or a combination thereof.

### Biodegradable Polymers

According to another embodiment, there is a biodegradable drug implant for reducing, preventing or treating radicular pain and/or inflammation in a patient in need of such treatment, the drug implant comprising dexamethasone in an amount of from 50% to about 80% by weight of the drug implant and a clonidine compound.

In some embodiments, the biodegradable polymer matrix comprises sustained release biopolymers including but not limited to poly (alpha-hydroxy acids), poly (lactide-co-glycolide) (PLGA), polylactide (PLA), polyglycolide (PG), polyethylene glycol (PEG) conjugates of poly (alpha-hydroxy acids), poly(orthoester)s (POE), poly(esteramide)s, polyaspirins, polyphosphagenes, starch, pre-gelatinized starch, hyaluronic acid, chitosans, gelatin, alginates, albumin, fibrin, vitamin E compounds, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof.

In some embodiments, the biodegradable polymer matrix comprises at least one biodegradable polymer comprises one or more of poly(lactide-co-glycolide) (PLGA), polylactide (PLA), polyglycolide (PGA), D-lactide, D,L-lactide, L-lactide, D,L-lactide-co-ε-caprolactone, L-lactide-co-ε-caprolactone, D,L-lactide-co-glycolide-co-ε-caprolactone, poly(D,L-lactide-co-caprolactone), poly(L-lactide-co-caprolactone), poly(D-lactide-co-caprolactone), poly(D,L-lactide), poly(D-lactide), poly(L-lactide), poly(esteramide) or a combination thereof.

In some embodiments, the biodegradable drug implant has a modulus of elasticity in the range of about 1×10² to about 6×10⁵ dyn/cm², or 2×10⁴ to about 5×10⁵ dyn/cm², or 5×10⁴ to about 5×10⁵ dyn/cm². In some embodiments, the biodegradable drug implant is in the form of a solid. In some embodiments, the biodegradable drug implant is solid or in semi-solid form. The solid or semi-solid form of the biodegradable drug implant may have a pre-dosed viscosity in the range of about 1 to about 2000 centipoise (cps), 1 to about 200 cps, or 1 to about 100 cps. After the solid or semi-solid biodegradable drug implant is administered to the site, the viscosity of the semi-solid or solid biodegradable drug implant will increase and the semi-solid will have a modulus of elasticity in the range of about 1×10² to about 6×10⁵ dynes/cm², or 2×10⁴ to about 5×10⁵ dynes/cm², or 5×10⁴ to about 5×10⁵ dynes/cm².

In various embodiments, the semi-solid or solid biodegradable drug implant may comprise a polymer (e.g., PLGA) having a molecular weight (MW), as shown by the inherent viscosity, from about 0.10 dL/g to about 1.2 dL/g or from about 0.20 dL/g to about 0.50 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.55 dL/g, about 0.50 to about 0.70 dL/g, about 0.55 to about 0.6 dL/g, about 0.60 to about 0.80 dL/g, about 0.70 to about 0.90 dL/g, about 0.80 to about 1.00 dL/g, about 0.90 to about 1.10 dL/g, about 1.0 to about 1.2 dL/g, about 1.1 to about 1.3 dL/g, about 1.2 to about 1.4 dL/g, about 1.3 to about 1.5 dL/g, about 1.4 to about 1.6 dL/g, about 1.5 to about 1.7 dL/g, about 1.6 to about 1.8 dL/g, about 1.7 to about 1.9 dL/g, or about 1.8 to about 2.1 dL/g.

In some embodiments, the biodegradable drug implant comprises one or more polymers having a MW of from about 15,000 to about 150,000 Da, from about 25,000 to about 100,000 Da or from about 30,000 to about 50,000 Da or about 30,000 Da to about 60,000 Da.

In some embodiments, the biodegradable drug implant comprises a polymer (e.g., PLGA) having an average molecular weight of the polymer can be from about 1,000 to about 10,000,000 Da; or about 1,000 to about 1,000,000 Da; or about 5,000 Da to about 500,000 Da; or about 10,000 Da to about 100,000 Da; or about 20,000 Da to 50,000 Da.

In some embodiments, when the polymer materials have different chemistries (e.g., high MW DLG 5050 and low MW DL), the high MW polymer may degrade faster than the low MW polymer.

Biodegradable drug implants made of dexamethasone and a polylactic acid polyglycolic acid (PLGA) copolymer matrix are provided which can release dexamethasone over various programmed time periods. When implanted at or near the target tissue and/or at or near the spine these biodegradable drug implants provide therapeutic levels of dexamethasone for reducing, preventing or treating radicular pain.

Accordingly, in one embodiment, a method for reducing, preventing or treating radicular pain in the spine of a patient is provided, the method comprising implanting at or near the spine a biodegradable drug implant comprising dexamethasone and a polylactic acid polyglycolic acid (PLGA) copolymer matrix, where the dexamethasone is released by erosion of the copolymer matrix. According to one embodiment, there is a method for treating radicular pain in a patient in need of such treatment, where said method comprises administering a biodegradable drug implant to a target tissue site beneath the skin of the patient, to reduce, prevent or treat radicular pain, wherein said implant comprises dexamethasone in an amount from about 50% to 80% of the weight of the drug implant, and a polylactic acid polyglycolic acid (PLGA) copolymer matrix, wherein the drug implant releases the dexamethasone over a period of at least about 30 days.

According to another embodiment, there is a method for treating radicular pain or inflammation including sciatica, wherein said implant comprises administering a biodegradable drug implant to a target tissue site beneath the skin of the patient, wherein said implant comprises dexamethasone in an amount from about 50% to 80% of the weight of the drug implant, and a polylactic acid polyglycolic acid (PLGA) copolymer matrix, wherein the drug implant releases the dexamethasone over a period of at least about 30 days.

The biodegradable drug implant may be implanted at various sites in the back, depending on the size, shape and formulation of the implant. In some embodiments, suitable sites include the epidural space, nerve root, dorsal root ganglion, focal sites of pain, at or near the spinal column. In a preferred embodiment, the biodegradable drug implant is placed at or near the target tissue in the spine.

In some embodiments, the biodegradable implants of the application are formulated with particles of drugs associated with the polylactic acid polyglycolic acid (PLGA) copolymer matrix. The rate of biodegradation is controlled by the ratio of glycolic to lactic acid. The percent of polylactic acid in the polylactic acid in the polylactic acid polyglycolic acid (PLGA) copolymer can be from about 1-100%, from about 15-85%, and from about 35-65%. In some embodiments, the percent of polylactic acid in the polylactic acid in the polylactic acid polyglycolic acid (PLGA) copolymer is from about 1%, 5%, 10%, 15%, 20%, 25%, 30%, 35%, 40%, 45%, 50%, 55%, 60%, 65%, 70%, 75%, 80%, 85%, 90%, 95% or 100%. In some embodiments, a 75/25, 50/50 or 25/75 PLGA copolymer is used. In various embodiments, the drug is entrapped within the PLGA copolymer matrix. Release of the drug is achieved by erosion of the polymer. Parameters which may determine the release kinetics include the size of the drug particles, the water solubility of the drug, the ratio of drug to polymer, the method of manufacture, the surface area exposed, and the erosion rate of the polymer.

In some embodiments, the drug comprises a corticosteroid or steroidal anti-inflammatory agent: beclomethasone, betamethasone, chloroprednisone, clobetasol, clobetasone, clocortolone, cloprednol, corticosterone, cortisone, dexamethasone, flucinolide, fluocinolone acetonide, fluticasone propionate, halobestasol propionate, hydrocortisone, methylprednisolone, mometasone furoate, prednisolone, prednisone, triamcinolone, triamcinolone acetonide, triamcinolone benetonide, and/or triamcinolone hexacetonide. In some embodiments, the drug is dexamethasone.

When referring to dexamethasone, unless otherwise specified or apparent from context it is understood that the inventors are also referring to pharmaceutically acceptable salts, pharmacologically active derivatives of the dexamethasone or an active metabolite of the dexamethasone. As used herein, “pharmaceutically acceptable salts” refer to derivatives of the disclosed compounds (e.g., esters or amines) wherein the parent compound may be modified by making acidic or basic salts thereof. Examples of pharmaceutically acceptable salts include, but are not limited to, mineral or organic acid salts of basic residues, such as amines; alkali or organic salts of acidic residues such as carboxylic acids. The pharmaceutically acceptable salts include the conventional non-toxic salts or quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, or nitric acids; or the salts prepared from organic acids such as acetic, fuoric, propionic, succinic, glycolic, stearic, lactic, malic, tartaric, citric, ascorbic, pamoic, maleic, hydromaleic, phenylacetic, glutamic, benzoic, salicylic, sulfanilic, 2-acetoxybenzoic, fumaric, tolunesulfonic, methanesulfonic, ethane disulfonic, oxalic, isethionic acid. Pharmaceutically acceptable also includes the racemic mixtures ((+)-R and (−)-S enantiomers) or each of the dextro and levo isomers of the dexamethasone individually. The dexamethasone may be in the free acid or base form or be pegylated for long acting activity.

### Additional Therapeutic Agents

In another embodiment, the biodegradable drug implant comprises more than one corticosteroid agent. The implant may further comprise one or more additional therapeutic agents, such as a clonidine compound, an analgesic, a GABA compound, a muscle relaxant, a non-steroidal anti-inflammatory compound, an anesthetic or a combination thereof.

In one embodiment, the additional therapeutic agent is a non-steroidal anti-inflammatory compound: aminoarylcarboxylic acid derivatives (e.g., flufenamic acid, meclofenamic acid), arylacetic acid derivatives (e.g., bromfenac, diclofenac sodium, indomethacin, tolmectin, zomepirac), arylbutyric acid derivatives (e.g., fenbufen), arylcarboxylic acids (e.g., ketorlac), arylpropionic acid derivatives (e.g., benoxaprofen, fenoprofen, flurbiprofen, ibuprofen, ketoprofen, naproxen, pirprofen), pyrazoles (e.g., difenamizole), pyrazoles (e.g., oxyphenbutazone, phenylbutazone), salicylic acid derivatives (e.g., aspirin, benorylate, diflunisal, salsalate), thiazinecarboxamides (e.g., piroxicam).

In one embodiment, the additional therapeutic agent is clonidine. In some embodiments, the clonidine is released locally from the biodegradable drug implant at a dose of from about 40 mcg/day for 7 days or between about 80 ug and 250 ug per day for a period of about 2 to 14 days about.

In one embodiment, the additional therapeutic agent is a GABA compound. In some embodiments, dexamethasone can be used with a GABA compound in the drug implant. The GABA compounds used in the treatment methods and in the device include compounds of gamma-aminobutyric acid. Such compounds include gabapentin (2-[1-(aminomethyl)cyclohexyl]acetic acid), pregabalin ((S)-3-(aminomethyl)-5-methylhexanoic acid), vigabatrin (4-aminohex-5-enoic acid), and baclofen (4-amino-3-(4-chlorophenyl)butanoic acid), which are 3′-alkylated GABA compounds. Additional GABA compounds that may be used are described in Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; WO 02/00209); Silverman et al., PCT Publication No. WO 92/09560; Silverman et al., PCT Publication No. WO 93/23383; Horwell et al., PCT Publication No. WO 97/29101, Horwell et al., PCT Publication No. WO 97/33858; Horwell et al., PCT Publication No. WO 97/33859; Bryans et al., PCT Publication No. WO 98/17627; Guglietta et al., PCT Publication No. WO 99/08671; Bryans et al., PCT Publication No. WO 99/21824; Bryans et al., PCT Publication No. WO 99/31057; WO 98/23383; Bryans et al., J. Med. Chem. 1998, 41, 1838-1845; Bryans et al., Med. Res. Rev. 1999, 19, 149-177, US Guglietta et al., WO 99/08670; Bryans et al., WO 99/21824; US Bryans et al., UK GB 2 374 595), Belliotti et al., PCT Publication No. WO 99/31074; Bryans et al., PCT Publication No. WO 99/31075; Bryans et al., PCT Publication No. WO 99/61424; Bryans et al., PCT Publication No. WO 00/15611; Bryans, PCT Publication No. WO 00/31020; Bryans et al., PCT Publication No. WO 00/50027; and Bryans et al., PCT Publication No. WO 02/00209). New classes of GABA compounds, which are bicyclic amino acid derivatives, have been recently described by Bryans et al., PCT Publication No. WO 01/28978; Blakemore et al., PCT Pub. No. WO 02/085839; Blakemore et al., U.S. Pat. No. 5,596,900; and Blakemore et al., PCT Pub. No. WO 02/090318. These disclosures are herein incorporated by reference into the present disclosure.

In one embodiment, the GABA compound comprises 1-{[(alpha-isobutanoyloxyethoxy)carbonyl]-aminomethyl}-1-cyclohexane acetic acid, baclofen, vigabatrin, gabapentin, pregabalin, gamma-amino-phosphinic acid or 1-{[(alpha-isobutanoyloxyethoxy)carbonyl]aminomethyl}-1-cyclohexane acetic acid, fengabine, GBL (gamma-Butyrolactone), GHB (gamma-Hydroxybutyric acid, 4-hydroxybutanoic acid or sodium oxybate), picamilon and progabide,(s)-(+)-4-amino-3-(2-methylpropyl) butanoic acid.

In another embodiment, GABA compounds include pharmaceuticals that can increase locally the available amount of endogenous GABA or GABA analogs following their local or systemic administration. These include pharmaceuticals that interfere with GABA or GABA analog reuptake such as tiagabine, stiripentol, deramciclane, hyperforin or a combination thereof. GABA compounds also include pharmaceuticals that interfere with the degradation of GABA or GABA analogs such as phenelzine, gabaculine, valproate, vigabatrin, lemon balm or a combination thereof.

In one embodiment, the additional therapeutic agent is a muscle relaxant. Exemplary muscle relaxants include, by way of example and not limitation, alcuronium chloride, atracurium bescylate, carbamate, carbolonium, carisoprodol, chlorphenesin, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

In another embodiment, the additional therapeutic agent is an analgesic. Suitable analgesic agents include, but are not limited to, acetaminophen, bupivacaine, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, meperidine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof.

In one embodiment, the additional therapeutic agent is an anesthetic: bupivacaine, liposomal bupivacaine, chloroprocaine, lidocaine, mepivacaine, prilocaine, procaine, and tetracaine.

In one embodiment, the implant delivers the dexamethasone for at least about 30 days. In other embodiments, the implant delivers the dexamethasone for at least about 3 weeks, at least about more than 30 days to at least about 60 days. In one embodiment, the implant delivers the dexamethasone for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 days. In one embodiment more than one biodegradable drug implant may be sequentially implanted into the spine to maintain drug concentration for even longer periods. In some embodiments, 1, 2, 3, 4, 5, 6, 7, 8, 9, 10 or more biodegradable drug implants may be sequentially implanted into a surgical site (e.g., spine).

According to another embodiment, there is a method for weight loss in a patient in need of such treatment, the method comprising administering a biodegradable drug implant to a target tissue site beneath the skin of the patient, the biodegradable drug implant comprising a wright losing amount of dexamethasone in an amount from about 50% to 80% of the weight of the drug implant, and a biodegradable polymer matrix, wherein the drug implant releases the dexamethasone over a period of at least about 30 days.

In some embodiments, the biodegradable polymer matrix is polylactic acid polyglycolic acid (PLGA) copolymer matrix. In some embodiments, the drug implant releases the dexamethasone over a period of at least about 3 weeks. In various embodiments, the weight loss occurs within one week after the implant is implanted beneath the skin and over a period of at least about 30 days. In some embodiments, the biodegradable drug implant is administered at or near the spine.

In some embodiments, the drug implant comprises a weight losing amount/weight control amount of dexamethasone in an amount from about 50 to 80% of the weight of the drug implant. In some embodiments, the biodegradable drug implant comprises a weight losing amount/weight control amount of dexamethasone in an amount from about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% to 80% of the weight of the drug implant. In some embodiments, the patient will exhibit about 1% to 30% weight loss from administration of the dexamethasone drug implant. In some embodiments, the patient will exhibit about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% weight loss from administration of the dexamethasone drug implant. In some embodiments, weight loss is determined by the patient's Body Mass Index (BMI) (BMI=mass(kg)/(height (m))² or mass(lb)/(height (in))²×703⁺), ideal weight losing amount and/or lean body weight (Body Weight−(Body Weight×Body Fat %).

In various embodiments, the drug implant comprises an anorectic, a corticosteroid, an anti-inflammatory agent, a stimulant or a combination thereof. In one embodiment, the additional therapeutic agent is an anorectic: lorcaserin, sibutramine, rimonabant, tesofensine topiramate, bupropion, naltrexone, zonisamide. In one embodiment, the additional therapeutic agent is a stimulant: phentermine, amphetamines (e.g., dexamphetamine, methamphetamine), diethylpropion, phenylpropanolamine.

The dexamethasone may also be administered with non-active ingredients. These non-active ingredients may have multi-functional purposes including the carrying, stabilizing and controlling the release of the therapeutic agent(s). The sustained release process, for example, may be by a solution-diffusion mechanism or it may be governed by an erosion-sustained process. In some embodiments, the implant (e.g., fiber, strip, pellet, etc.) will be a solid or semi-solid formulation comprised of a biocompatible material that can be biodegradable.

Exemplary excipients, plasticizers, and/or pore forming agents that may be formulated with dexamethasone in addition to the biodegradable polymer include but are not limited to MgO (e.g., 1 wt. %), mPEG, propylene glycol, mannitol, trehalose, TBO-Ac, Span-65, Span-85, pluronic F127, sorbitol, cyclodextrin, maltodextrin, pluronic F68, CaCl, dextran, dextran sulphate, dextran phosphate, hydroxypropylcellulose, ethylcellulose, PEG 1500, PEG 400, PEG3350 or combinations thereof.

In some embodiments, the excipients comprise from about 0.001 wt. % to about 50 wt. % of the formulation. In some embodiments, the excipients comprise from about 0.001 wt. % to about 40 wt. % of the formulation. In some embodiments, the excipients comprise from about 0.001 wt. % to about 30 wt. % of the formulation. In some embodiments, the excipients comprise from about 0.001 wt. % to about 20 wt. % of the formulation. In some embodiments, the excipients comprise from about 0.001 wt. % to about 10 wt. % of the formulation. In some embodiments, the excipients comprise from about 0.001 wt. % to about 50 wt. % of the formulation. In some embodiments, the excipients comprise from about 0.001 wt. % to about 2 wt. % of the formulation.

### EXAMPLES

The examples below with respect to certain formulations comprising dexamethasone as the biologically active agent show certain particularly advantageous results.

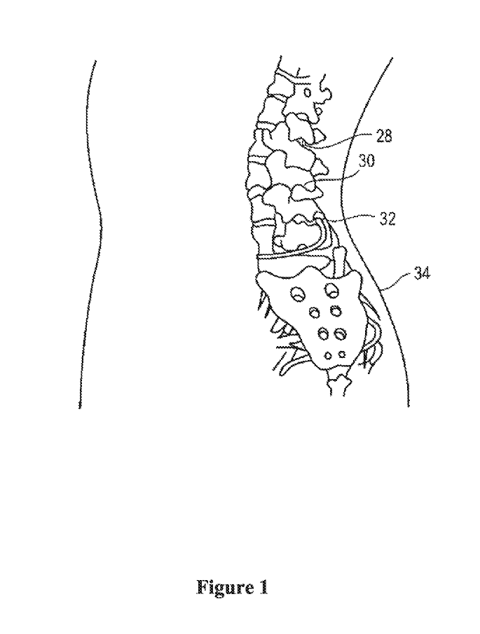

In one embodiment, as shown in FIG. 1, the dexamethasone drug implant/s are suitable for use in pain management (e.g., neuropathic pain management) and/or to treat conditions (e.g., sciatica). FIG. 1 is a dorsal view of the spine and sites where the dexamethasone drug implant's (e.g., depot/s) (containing the sustained release clonidine) may be inserted using a cannula or needle beneath the skin 34 to a spinal site 32 (e.g., spinal disc space, spinal canal, soft tissue surrounding the spine, nerve root, etc.) and one or more drug implants 28 and 30 are delivered to various sites along the spine. In this way, when the dexamethasone drug implant/s are implanted, they are implanted in a manner that optimizes location, accurate spacing, and drug distribution. In some embodiments, the dexamethasone drug implant/s can be administered with one needle. In some embodiments, the dexamethasone drug implant/s can be administered using a subpedicular or retroneural technique.

### Example 1

### Effectiveness of Dexamethasone 0.7 mg Implant Dexamethasone (Ozurdex®) in Neuropathic Pain in Rat Model

The purpose of this 57-day study was to determine whether Ozurdex® dexamethasone-eluting pellets can improve pain-associated behavioral responses in a rat model of neuropathic pain one week after injury and sustained for two months. An additional purpose was to compare the effects of Ozurdex® dexamethasone-eluting pellets to 8% or 15% clonidine/PLA formulations.

Pre-operative baseline thresholds for thermal hyperalgesia and mechanical allodynia were obtained for male Sprague-Dawley rats. The animals were then subjected to CCI surgery, and randomly assigned to treatment groups. Over the course of the study, the effectiveness of each treatment was determined by thermal nociceptive and mechanical tactile allodynia tests.

Using the rat chronic constriction injury (CCI) model under 2% isoflurane anesthesia, an incision was made on the right, lateral side (mid-thigh) of each, and the common sciatic nerve was exposed and freed from adherent tissue by separating the muscle (biceps femoris) by blunt dissection. Four loose chronic gut ligatures (4-0 absorbable sutures, Jorgensen Laboratories, Inc., Loveland, Colo.) were tied around the nerve approximately 1 mm apart.

Beginning on Day 7 post-surgery, animals received local doses of clonidine-loaded pellets (8% and 15% PLA) (Medtronic), unloaded PLA pellets (solid rod) depots (Medtronic) and Ozurdex® dexamethasone-loaded pellets (0.7 mg) (Allergan) were implanted imtramuscularly adjacent and parallel to the injured sciatic nerve in Sprague Dawley rats as near as possible to the nerve without actually touching it. Two groups of animals also received systemic clonidine and dexamethasone administered subcutaneously (sc) at a dose of 0.02 mg/kg every day starting at day 7 post-surgery. Pain-associated behaviors were tested using the Thermal Paw Withdrawal Latency Test and the von Frey Monofilament Test.

Systemic dexamethasone significantly improved thermal paw withdrawal responses following a thermal stimulus on various test days compared with both PBS and unloaded PLA pellet (p<0.05). However, systemic dexamethasone administered sc had no significant effect on mechanical allodynia relative to either PBS or unloaded pellet.

### Behavioral Tests

Thermal Paw Withdrawal Latency Test.

The method of Hargreaves et al. (1988) was used to assess paw-withdrawal latency to a thermal nociceptive stimulus using a standard plantar analgesia instrument (Stoelting, Wood Dale, Ill.). Prior to testing, each animal was placed on the plantar test apparatus, a clear plastic chamber, and allowed to rest/acclimate for 15 min. A radiant (heat) beam stimulus was applied to the CCI hind paw of each animal. Paw withdrawal stopped both the stimulus and the timer. The heat source device was set at intensity 50, and a maximal cut-off of 15s was set to prevent tissue damage. Thermal hyperalgesia paw withdrawal latency response of the injured paw of each animal was measured on days −1 (pre-injury baseline), 7 (before treatment), 7 (4h post-administration), 14, 21, 28, 35, 42, 49, and 56.

Tactile Allodynia Test.

Mechanical allodynia was measured using the calibrated von Frey Monofilament Test (Stoelting, Wood Dale, Ill.). The plantar surface of the injured paw of each animal was tested as described by Chaplan et al. (1994). Each animal was placed in a suspended clear plastic chamber with a wire mesh bottom. Prior to testing, each animal was acclimated for 15 min. The 50% paw withdrawal threshold response was determined by sequentially increasing or decreasing the stimulus strength according to the “up-down method” of Dixon (1980). Testing began with a filament with a buckling weight of 2.0 g, and continued through a series of filaments applied in sequence up to a buckling weight of 15 g. Each filament was applied with enough pressure to cause a buckle effect. The absence of a paw lifting/withdrawal response after 5 s prompted the use of the next higher weight filament. Paw withdrawal, indicating a positive response, prompted the use of a weaker filament. After the initial positive response (e.g., paw withdrawal), the testing continued for four additional measurements, and was used to calculate the response threshold. Four consecutive positive responses received a score of 0.25 g, and five consecutive negative responses (e.g., no paw withdrawal) received a score of 15 g. The mechanical paw withdrawal threshold of each animal was measured on days −1 (pre-injury baseline), 8, 15, 22, 29, 36, 43, 50, and 57.

Analyses for Tactile Allodynia Testing.

The 50% paw withdrawal threshold was calculated (PWT; Luo and Calcutt, 2002; Chaplan et al., 1994) using the formula: 10 (Xf+K6)/10,000 where Xf is the final von Frey filament used (log units), lc is a value that analyzes the response pattern (taken from the table published by Chaplan et al, 1994), and 6 is the mean difference between stimuli (log units).

Statistical Analyses.

Analysis of variance (ANOVA) was used to assess the effect of each treatment group as compared to PBS. Drug treatment was the between group variable, whereas the test day was the repeated measure. Since overall ANOVA produced significant effects, one-way ANOVA was performed for each test day. Group differences were determined using Fisher's PLSD tests as post-hoc analyses. The p-value for significance was set at p<0.05.

### Results

From Day 7 through Day 35, it was observed that the Ozurdex® implant Groups exhibited notable body weight loss. By day 35 however, body weights became stable and the animals gained weight for the remainder of the study.

No overt side effects or adverse reactions at the site of injection occurred in any of the animals treated with the clonidine, Ozurdex®, or unloaded pellets; nor the systemic delivery of clonidine, dexamethasone, or PBS over the 57-day duration of the study. The adverse effects observed in the Ozurdex® treatment groups were limited to the body weight loss.

At the end of the study, gross necropsies were conducted. No polymer pellet traces were found in the animals treated with Ozurdex®.

Ozurdex® Pellet.

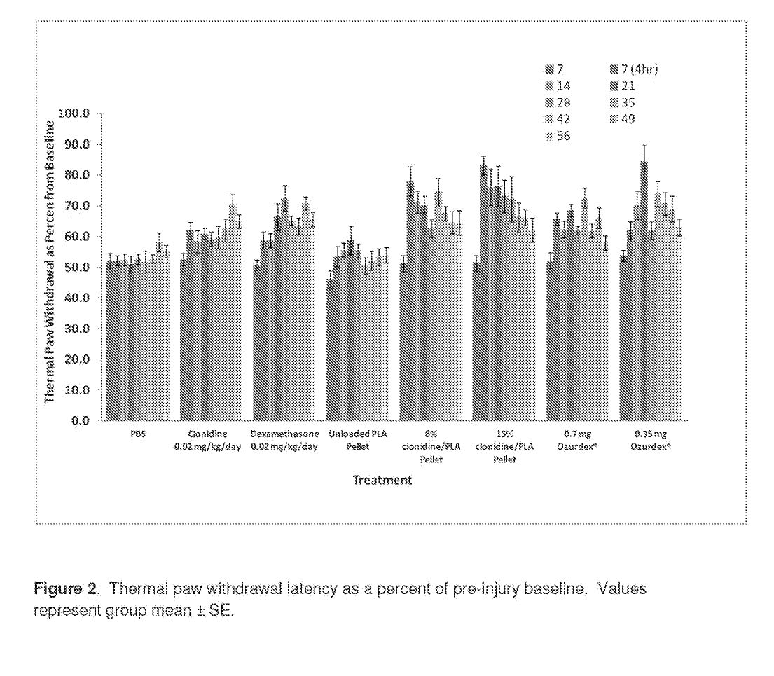

Data for thermal testing of the animals treated with a single Ozurdex® pellet are shown in Table 1 and FIG. 2. The overall ANOVA indicated that the Ozurdex® pellet showed a significant decrease in thermal hyperalgesia when compared with PBS [F (1,12)=28.5, p<0.05]. The one-way ANOVA indicated a significant effect of the Ozurdex® pellet on day 7 (4 h post-administration) [F (1,12)=47.7, p<0.05], day 14 [F (1,12)=8.1, p<0.05], day 21 [F (1,12)=23.1, p<0.05], day 28 [F (1,12)=19.1, p<0.05], day 35 [F (1,12)=21.4, p<0.05] and day 42 [F (1,12)=8.1, p<0.05]. However, the Ozurdex® pellet had no effect on thermal hyperalgesia on day 7 (pre-treatment) [F (1,12)=0.0], day 49 [F (1,12)=3.6] and day 56 [F (1,12)=0.9]. Post-hoc analyses revealed that the Ozurdex® pellet produced a significant increase in thermal latency only on days 7 (4 h post-administration), 14, 21, 28, 35 and 42 relative to PBS (Fisher PLSD, p<0.05).

Data for thermal testing of the animals treated with the Ozurdex® pellet were also compared with unloaded PLA pellets. The overall ANOVA indicated that the Ozurdex® pellet showed a significant decrease in thermal hyperalgesia when compared with unloaded PLA pellet [F (1,14)=27.6, p<0.05]. The one-way ANOVA indicated a significant effect of the Ozurdex® pellet on day 7 (4 h post-administration) [F (1,14)=15.1, p<0.05], day 28 [F (1,14)=5.9, p<0.05], day 35 [F (1,14)=32. 2, p<0.05], day 42 [F (1,14)=7.8, p<0.05] and day 49 [F (1,14)=8.7, p<0.05]. However, the Ozurdex® pellet had no effect on thermal hyperalgesia on day 7 (pre-treatment) [F (1,14)=2.3], day 14 [F (1,14)=3.6], day 21 [F (1,14)=4.3], and day 56 [F (1,14)=1.0]. Post-hoc analyses revealed that the Ozurdex® pellet produced a significant increase in thermal latency only on days 7 (4 h post-administration), 28, 35, 42 and 49 relative to unloaded PLA pellet (Fisher PLSD, p<0.05).

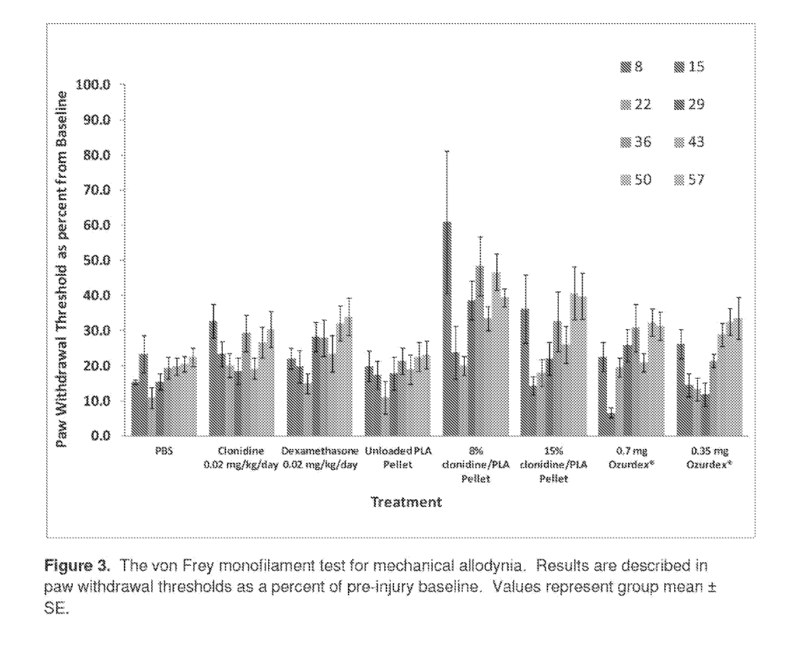

Data for mechanical allodynia of the animals treated with the Ozurdex® pellet is shown in Table 1 and FIG. 3. The overall ANOVA indicated that the Ozurdex® pellet produced no effect on mechanical allodynia relative to PBS [F (1,12)=2.2] or unloaded PLA pellet [F (1,14)=2.2].

TABLE 1
Table 1. Ozurdex ® Pellet Behavioral Testing Results

| | |
|---|---|
| PBS | Unloaded PLA Pellet |

Thermal Hyperalgesia

| | | | |
|---|---|---|---|
| Day 7 | Mean | 52.2 | |
| | S.E. | 2.6 | |
| | T-test | t(13) = 20.9 | t(15) = 22.55 |
| Day 7 (4 h) | Mean | 65.7 | |
| | S. E. | 2.0 | |
| | T-test | t(13) = 14.92* | t(15) = 14.86* |
| Day 14 | Mean | 62.2 | |
| | S. E. | 2.7 | |
| | T-test | t(13) = 16.47* | t(15) = 19.23 |
| Day 21 | Mean | 68.5 | |
| | S. E. | 2.1 | |
| | T-test | t(13) = 12.42* | t(15) = 12.73 |
| Day 28 | Mean | 62.0 | |
| | S.E. | 1.3 | |
| | T-test | t(13) = 20.08* | t(15) = 22.74* |
| Day 35 | Mean | 72.5 | |
| | S. E. | 3.2 | |
| | T-test | t(13) = 9.16* | t(15) = 10.70* |
| Day 42 | Mean | 61.8 | |
| | S. E. | 2.3 | |
| | T-test | t(13) = 19.89* | t(15) = 16.83* |
| Day 49 | Mean | 66.0 | |
| | S. E. | 3.3 | |
| | T-test | t(13) = 12.60 | t(15) = 14.45* |
| Day 56 | Mean | 57.8 | |
| | S. E. | 2.4 | |
| | T-test | t(13) = 20.59 | t(15) = 22.54 |

Mechanical Allodynia

| | | | |
|---|---|---|---|
| Day 8 | Mean | 22.5 | |
| | S. E. | 4.1 | |
| | T-test | t(13) = 9.43 | t(15) = 9.31 |
| Day 15 | Mean | 6.6 | |
| | S. E. | 1.4 | |
| | T-test | t(13) = 9.20 | t(15) = 10.64 |
| Day 22 | Mean | 19.6 | |
| | S.E. | 2.7 | |
| | T-test | t(13) = 9.18 | t(15) = 10.23 |
| Day 29 | Mean | 25.8 | |
| | S.E. | 4.6 | |
| | T-test | t(13) = 9.42 | t(15) = 9.53 |
| Day 36 | Mean | 30.8 | |
| | S.E. | 6.7 | |
| | T-test | t(13) = 8.03 | t(15) = 8.60 |
| Day 43 | Mean | 20.8 | |
| | S.E. | 2.7 | |
| | T-test | t(13) = 9.85 | t(15) = 9.96 |
| Day 50 | Mean | 32.2 | |
| | S.E. | 3.9 | |
| | T-test | t(13) = 9.04 | tt(15) = 9.26 |
| Day 57 | Mean | 31.4 | |
| | S.E. | 4.0 | |
| | T-test | t(13) = 8.92 | t(15) = 9.30 |

*p < 0.05 when compared to PBS or unloaded PLA pellet

The data from this study indicated no significant difference with regard to thermal hyperalgesia between the groups treated with PBS and unloaded PLA pellet. Also, the data from this study indicated no significant difference with regard to mechanical allodynia between the groups treated with PBS and unloaded PLA pellet.

Data from this study indicated that administration of one 8% clonidine pellet significantly increased thermal paw withdrawal latencies on various test days relative to both PBS and unloaded PLA (p<0.05). Also, the 8% clonidine pellet significantly improved mechanical allodynia on various test days relative to both PBS and unloaded PLA pellet (p<0.05).

The results of this study also indicated that administration of one 15% clonidine pellet significantly increased thermal paw withdrawal latencies on all test days, except for day 56, relative to both PBS and unloaded pellet (p<0.05). Also, the 15% clonidine pellet significantly improved mechanical allodynia on various test days relative to both PBS and unloaded PLA pellet (p<0.05).

The Ozurdex® (0.7 mg dexamethasone) eluting pellets significantly increased thermal paw withdrawal latencies on various test days relative to both PBS and unloaded PLA pellet (p<0.05). However, Ozurdex® had no significant effect on mechanical allodynia relative to either the PBS or unloaded pellet.

There were no overt side effects or adverse reactions at the site of injection in any of the animals treated systemically with clonidine, dexamethasone, or PBS over the duration of this study. In addition, no overt side effects or adverse reactions were seen at the site of implantation in any of the animals treated locally with clonidine or unloaded PLA pellet. In contrast, local delivery of Ozurdex® administered at doses of ˜0.35 mg and 0.7 mg caused notable body weight loss that was observed during the second week of the 8 week study. No adverse reactions at the site of implantation occurred in any of the animals treated with Ozurdex®.

In conclusion, this study demonstrated that local doses of clonidine or Ozurdex® administered by biodegradable polymers were effective for reducing thermal hyperalgesia in the CCI rat model of neuropathic pain, but only the 8% and 15% clonidine pellets were effective at reducing mechanical allodynia. Prolonged delivery of clonidine and Ozurdex® via locally applied polymer pellets may offer a feasible alternative to commercially available steroidal and non-steroidal anti-inflammatory drugs for neuropathic pain.

### Example 2

### Effectiveness of Dexamethasone Half Pellet (0.35 mg) Implant (Ozurdex®) in Neuropathic Pain Rat Model

The half-pellet dose of Ozurdex® (˜0.35 mg) was significantly increased thermal paw withdrawal latencies on various test days relative to both PBS and unloaded pellet (p<0.05). However, the half-pellet dose had no significant effect on mechanical allodynia relative to either PBS or unloaded PLA pellet.

Half of an Ozurdex® Pellet.

Data for thermal testing of the animals treated with a half pellet dose of Ozurdex® (˜0.35 mg dexamethasone) are shown in Table 2 and FIG. 2. The overall ANOVA indicated that half of an Ozurdex® pellet showed a significant decrease in thermal hyperalgesia when compared with PBS [F (1,12)=45.5, p<0.05]. The one-way ANOVA indicated a significant effect of half of an Ozurdex® pellet on day 7 (4 h post-administration) [F (1,12)=8.2, p<0.05], day 14 [F (1,12)=13.3, p<0.05], day 21 [F (1,12)=28.9, p<0.05], day 28 [F (1,12)=7.3, p<0.05], day 35 [F (1,12)=21.9, p<0.05], day 42 [F (1,12)=18.2, p<0.05] and day 56 [F (1,12)=6.2, p<0.05]. However, half of an Ozurdex® pellet had no effect on thermal hyperalgesia on day 7 (pre-treatment) [F (1,12)=0.2] and day 49 [F (1,12)=4.6]. Post-hoc analyses revealed that half of an Ozurdex® pellet produced a significant increase in thermal latency on days 7 (4 h post-administration), 14, 21, 28, 35, 42 and 56 relative to PBS (Fisher PLSD, p<0.05).

Data for thermal testing of the animals treated with half of an Ozurdex® pellet were also compared with unloaded PLA pellets. The overall ANOVA indicated that half of an Ozurdex® pellet showed a significant decrease in thermal hyperalgesia when compared with unloaded PLA pellet [F (1,14)=46.3, p<0.05]. The one-way ANOVA indicated a significant effect of half of an Ozurdex® pellet on day 14 [F (1,14)=9.8, p<0.05], day 21 [F (1,14)=15.2, p<0.05], day 35 [F (1,14)=33.0, p<0.05], day 42 [F (1,14)=19.3, p<0.05], day 49 [F (1,14)=10.2, p<0.05] and day 56 [F (1,14)=5.5, p<0.05]. However, half of an Ozurdex® pellet had no effect on thermal hyperalgesia on day 7 (pre-treatment) [F (1,14)=4.2], day 7 (4 h post-administration) [F (1,14)=3.7] and day 28 [F (1,14)=2.5]. Post-hoc analyses revealed that half of an Ozurdex® pellet produced a significant increase in thermal latency only on days 14, 21, 35, 42, 49 and 56 relative to unloaded PLA pellet (Fisher PLSD, p<0.05).

Data for mechanical allodynia of the animals treated with half of an Ozurdex® pellet are shown in Table 2 and FIG. 3. The overall ANOVA indicated half of an Ozurdex® pellet produced no effect on mechanical allodynia relative to PBS [F (1,12)=0.8] or unloaded PLA pellet [F (1,14)=0.6].

TABLE 2
Table 2. Half of an Ozurdex ® Pellet Behavioral Testing Results

| | |
|---|---|
| PBS | Unloaded PLA Pellet |

Thermal Hyperalgesia

| | | | |
|---|---|---|---|
| Day 7 | Mean | 53.7 | |
| | S.E. | 1.8 | |
| | T-test | t(13) = 21.06 | t(15) = 21.64 |
| Day 7 (4 h) | Mean | 61.9 | |
| | S.E. | 2.9 | |
| | T-test | t(13) = 15.66* | t(15) = 15.59 |
| Day 14 | Mean | 70.3 | |
| | S.E. | 4.5 | |
| | T-test | t(13) = 9.64* | t(15) = 11.31* |
| Day 21 | Mean | 84.3 | |
| | S.E. | 5.5 | |
| | T-test | t(13) = 5.23* | t(15) = 5.88* |
| Day 28 | Mean | 61.9 | |
| | S.E. | 2.8 | |
| | T-test | t(13) = 16.03* | t(15) = 18.19 |
| Day 35 | Mean | 73.9 | |
| | S.E. | 4.1 | |
| | T-test | t(13) = 8.03* | t(15) = 9.42* |
| Day 42 | Mean | 70.7 | |
| | S.E. | 3.7 | |
| | T-test | t(13) = 10.52* | t(15) = 10.55* |
| Day 49 | Mean | 69.0 | |
| | S.E. | 4.3 | |
| | T-test | t(13) = 9.54 | t(15) = 11.07* |
| Day 56 | Mean | 63.1 | |
| | S.E. | 2.7 | |
| | T-test | t(13) = 14.93* | t(15) = 16.38* |

Mechanical Allodynia

| | | | |
|---|---|---|---|
| Day 8 | Mean | 26.3 | |
| | S.E. | 4.2 | |
| | T-test | t(13) = 9.10 | t(15) = 8.93 |
| Day 15 | Mean | 14.5 | |
| | S.E. | 3.3 | |
| | T-test | t(13) = 9.01 | t(15) = 10.26 |
| Day 22 | Mean | 13.3 | |
| | S.E. | 3.2 | |
| | T-test | t(13) = 9.82 | t(15) = 10.84 |
| Day 29 | Mean | 11.8 | |
| | S.E. | 3.4 | |
| | T-test | t(13) = 10.85 | t(15) = 10.67 |
| Day 36 | Mean | 21.4 | |
| | S.E. | 1.9 | |
| | T-test | t(13) = 9.86 | t(15) = 10.09 |
| Day 43 | Mean | 28.9 | |
| | S.E. | 3.3 | |
| | T-test | t(13) = 9.22 | t(15) = 9.20 |
| Day 50 | Mean | 32.5 | |
| | S.E. | 3.8 | |
| | T-test | t(13) = 8.88 | tt(15) = 9.04 |
| Day 57 | Mean | 33.5 | |
| | S.E. | 6.0 | |
| | T-test | t(13) = 8.43 | t(15) = 8.79 |

*p < 0.05 when compared to PBS or unloaded PLA pellet

In this rat CCI model, clonidine- and Ozurdex® (dexamethasone)-loaded and unloaded PLA pellets were implanted in the epineural space; the individual pellets were placed caudal and parallel to the injured nerve (e.g., ipsilateral implant). Systemic clonidine and dexamethasone injections were administered sc at a dose of 0.02 mg/kg every day, and did not cause any overt side effects or adverse reactions in the animals; neither did the 8% clonidine pellet, 15% clonidine pellet, or an unloaded PLA pellet. However, Ozurdex®-treated rats had considerable weight loss observed within two weeks of administration. The weight loss eventually resolved. Pain-associated behaviors were tested using the Thermal Paw Withdrawal Latency Test and the von Frey Monofilament Test.

The data from this 57-day study indicated that all treatments including systemic delivery of clonidine and dexamethasone, 8% and 15% clonidine pellets and 0.7 mg and −0.35 mg Ozurdex®-eluting pellets increased thermal paw withdrawal latencies on various test days relative to both PBS and unloaded PLA pellet (p<0.05). Both 8% and 15% clonidine pellets significantly improved mechanical allodynia. However, the other treatment groups (groups treated with systemic injections of clonidine and dexamethasone and those treated with 0.7 mg and −0.35 mg Ozurdex® pellets) had no effect on paw withdrawal threshold responses relative to either PBS or unloaded PLA pellet.

The present study demonstrated that local doses of clonidine or Ozurdex® administered by biodegradable polymers are effective in reducing thermal hyperalgesia in the CCI rat model of neuropathic pain, but only the 8% and 15% clonidine pellets were also effective at reducing mechanical allodynia on various test days. With the exception of marked weight loss early in the study by the Ozurdex®-treated groups, these prolonged-release implants appear to be a safe and effective way of providing long-lasting analgesia, and, thus, offer a novel approach for treating neuropathic pain in humans.

### Example 3

### Ozurdex® Pellets Vs Systemic Clonidine Injections

Data for thermal testing of the animals treated with an Ozurdex® pellet were compared with systemic clonidine injections. The overall ANOVA indicated that the Ozurdex pellet showed no significant decrease in thermal hyperalgesia when compared with systemic clonidine injections (0.02 mg/kg/day) [F (1,12)=2.7].

Data for thermal testing of the animals treated with a half-pellet of Ozurdex® were compared with systemic clonidine injections. The overall ANOVA indicated that half of an Ozurdex® pellet caused a significant decrease in thermal hyperalgesia when compared with systemic clonidine injections [F (1,12)=11.2, p<0.05]. The one-way ANOVA indicated a significant effect of half of an Ozurdex® pellet on day 14 [F (1,12)=5.0, p<0.05], day 21 [F (1,12)=16.7, p<0.05] and day 35 [F (1,12)=11.7, p<0.05]. Post-hoc analyses revealed that half of an Ozurdex® pellet produced a significant increase in thermal latency only on days 14, 21 and 35 relative to systemic clonidine injections (Fisher PLSD, p<0.05).

Data for mechanical allodynia of the animals treated with an Ozurdex® pellet were also compared with systemic clonidine injections. The overall ANOVA indicated that the Ozurdex® pellet produced no effect on mechanical allodynia relative to systemic clonidine injections [F (1,12)=0.02].

Data for mechanical allodynia of the animals treated with half of an Ozurdex® pellet were also compared with systemic clonidine injections. The overall ANOVA indicated that half of an Ozurdex® pellet produced no effect on mechanical allodynia relative to systemic clonidine injections [F (1,12)=0.1].

### Example 4

### Ozurdex® Pellet Vs. Systemic Dexamethasone Injections

Data for thermal testing of the animals treated with an Ozurdex® pellet were compared with systemic dexamethasone injections (0.02 mg/kg/day). The overall ANOVA indicated that the Ozurdex® pellet showed no significant decrease in thermal hyperalgesia when compared with systemic dexamethasone injections [F (1,14)=0.2].

Data for thermal testing of the animals treated with half of an Ozurdex® pellet were compared with systemic dexamethasone injections. The overall ANOVA indicated that half of an Ozurdex® pellet caused no significant difference in thermal hyperalgesia when compared with systemic dexamethasone injections [F (1,14)=3.9].

Data for mechanical allodynia of the animals treated with the Ozurdex® pellet were also compared with systemic dexamethasone injections. The overall ANOVA indicated that an Ozurdex® pellet showed no effect on mechanical allodynia when compared with systemic dexamethasone injections [F (1,14)=1.8].

Data for mechanical allodynia of the animals treated with half of an Ozurdex® pellet were compared with systemic dexamethasone injections. The overall ANOVA indicated that half of an Ozurdex® pellet showed no effect on mechanical allodynia when compared with systemic dexamethasone injections [F (1,14)=0.4].

### Example 5

### Ozurdex® Pellet Vs Half of an Ozurdex® Pellet

Data for thermal testing of the animals treated with an Ozurdex® pellet were compared with half of an Ozurdex® pellet. The overall ANOVA showed no difference between the two treatments [F (1,14)=2.6].

Data for mechanical allodynia of the animals treated with an Ozurdex® pellet were compared with half of an Ozurdex® pellet. The overall ANOVA showed no difference between the two treatments [F (1,14)=0.2].

### Example 6

### Ozurdex® Pellet Vs. Clonidine Pellet

Data for thermal testing of the animals treated with an Ozurdex® pellet were compared with the 8% clonidine pellet. The overall ANOVA indicated that the Ozurdex® pellet showed no significant decrease in thermal hyperalgesia when compared with the 8% clonidine pellet [F (1,14)=2.7].

Data for thermal testing of the animals treated with the Ozurdex® pellet were compared with the 15% clonidine pellet. The overall ANOVA indicated that the Ozurdex® pellet showed no decrease in thermal hyperalgesia when compared with the 15% clonidine pellet [F (1,14)=4.5].

Data for mechanical allodynia of the animals treated with an Ozurdex® pellet were compared with the 8% clonidine pellet. The overall ANOVA indicated that the Ozurdex® pellet showed no effect in mechanical allodynia when compared with the 8% clonidine pellet [F (1,14)=0.5].

Data for mechanical allodynia of the animals treated with an Ozurdex® pellet were compared with the 15% clonidine pellet. The overall ANOVA indicated that the Ozurdex® pellet showed no effect in mechanical allodynia when compared with the 15% clonidine pellet [F (1,14)=2.6].

### Example 7

### Half of an Ozurdex® Pellet Vs Clonidine Pellet

Data for thermal testing of the animals treated with half of an Ozurdex® pellet were compared with the 8% clonidine pellet. The overall ANOVA indicated that half of an Ozurdex® pellet showed no significant decrease in thermal hyperalgesia when compared with the 8% clonidine pellet [F (1,14)=0.04].

Data for thermal testing of the animals treated with half of an Ozurdex® pellet were compared with the 15% clonidine pellet. The overall ANOVA indicated that half of an Ozurdex® pellet showed no significant decrease in thermal hyperalgesia when compared with the 15% clonidine pellet [F (1,14)=0.5].

Data for mechanical allodynia of the animals treated with half of an Ozurdex® pellet were compared with the 8% clonidine pellet. The overall ANOVA indicated that half of an Ozurdex® pellet showed no effect in mechanical allodynia when compared with the 8% clonidine pellet [F (1,14)=1.2].

Data for mechanical allodynia of the animals treated with half of an Ozurdex® pellet were compared with the 15% clonidine pellet. The overall ANOVA indicated that half of an Ozurdex® pellet showed no effect in mechanical allodynia when compared with the 15% clonidine pellet [F (1,14)=3.6].

### Example 8

### Ozurdex® Pellet Vs Clonidine Pellet

Data for thermal testing of the animals treated with an Ozurdex® pellet on day 7 (4 h post-administration) were compared with the 8% clonidine pellet on day 7 (4 h post-administration). The one-way ANOVA indicated that the 8% clonidine pellet on day 7 (4 h post-administration) showed a significant decrease in thermal hyperalgesia when compared with the Ozurdex® pellet on day 7 (4 h post-administration) [F (1,14)=9.3, p<0.05].

Data for thermal testing of the animals treated with half of an Ozurdex® pellet on day 7 (4 h post-administration) were compared with the 8% clonidine pellet on day 7 (4 h post-administration). The one-way ANOVA indicated that the 8% clonidine pellet on day 7 (4 h post-administration) showed a significant decrease in thermal hyperalgesia when compared with half of an Ozurdex® pellet on day 7 (4 h post-administration) [F (1,14)=14.0, p<0.05].

Data for thermal testing of the animals treated with an Ozurdex® pellet on day 7 (4 h post-administration) were compared with the 15% clonidine pellet on day 7 (4 h post-administration). The one-way ANOVA indicated that the 15% clonidine pellet on day 7 (4 h post-administration) showed a significant decrease in thermal hyperalgesia when compared with the Ozurdex® pellet on day 7 (4 h post-administration) [F (1,14)=75.2, p<0.05].

Data for thermal testing of the animals treated with half of an Ozurdex® pellet on day 7 (4 h post-administration) were compared with the 15% clonidine pellet on day 7 (4 h post-administration). The one-way ANOVA indicated that the 15% clonidine pellet on day 7 (4 h post-administration) showed a significant decrease in thermal hyperalgesia when compared with half of an Ozurdex® pellet on day 7 (4 h post-administration) [F (1,14)=54.8, p<0.05].

### Example 9

### Ozurdex® Pellet Vs Systemic Clonidine

Data for thermal testing of the animals treated with an Ozurdex® pellet on day 7 (4 h post-administration) were compared with systemic clonidine (0.02 mg/kg/day) on day 7 (4 h post-administration). The one-way ANOVA indicated no difference in thermal hyperalgesia between the two treatment groups on day 7 (4 h post-administration) [F (1,12)=2.7].

Data for thermal testing of the animals treated with half of an Ozurdex® pellet on day 7 (4 h post-administration) were compared with systemic clonidine on day 7 (4 h post-administration). The one-way ANOVA indicated no difference in thermal hyperalgesia between the two treatment groups on day 7 (4 h post-administration) [F (1,12)=0.0].

### Example 10

### Ozurdex® Pellet Vs Systemic Dexamethasone

Data for thermal testing of the animals treated with an Ozurdex® pellet on day 7 (4 h post-administration) were compared with systemic dexamethasone on day 7 (4 h post-administration). The one-way ANOVA indicated that the Ozurdex® pellet on day 7 (4 h post-administration) showed a significant decrease in thermal hyperalgesia when compared with systemic dexamethasone on day 7 (4 h post-administration) [F (1,14)=10.2, p<0.05].

Data for thermal testing of the animals treated with half of an Ozurdex® pellet on day 7 (4 h post-administration) were compared with systemic dexamethasone on day 7 (4 h post-administration). The one-way ANOVA indicated no significant difference in thermal hyperalgesia between the two treatment groups on day 7 (4 h post-administration) [F (1,14)=1.0].

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

Having now generally described the application, the same may be more readily understood through the following reference to the following examples, which are provided by way of illustration and are not intended to limit the present application unless specified.

1. A biodegradable drug implant for reducing, preventing or treating radicular pain and/or inflammation in a patient in need of such treatment, the biodegradable drug implant comprising dexamethasone and a polylactic acid polyglycolic acid (PLGA) copolymer matrix, wherein the dexamethasone comprises from about 50% to about 80% by weight of the drug implant, the implant comprises a pellet having a burst release surface that releases a bolus dose of the dexamethasone that is 5% to 15% of the dexamethasone loaded in the drug implant within 24 hours and releases the remaining dexamethasone over a period of at least about 30 days.

2. A biodegradable drug implant of claim 1, wherein the drug implant is configured for placement under the skin.

3. A biodegradable drug implant of claim 1, wherein the dexamethasone comprises about 60% to 70% of the weight of the drug implant.

4. A biodegradable drug implant of claim 2, wherein the drug implant releases the dexamethasone in an amount of about 0.1 mg to 4 mg over a period of 30 to 60 days after placement beneath the skin.

5. A biodegradable drug implant of claim 2, wherein the drug implant releases the dexamethasone in an amount of 0.35 mg over a period of 30 to 60 days after placement beneath the skin.

6. A biodegradable drug implant of claim 2, wherein the drug implant releases the dexamethasone in an amount of 0.7 mg over a period of 30 to 60 days after placement beneath the skin.

7. A biodegradable implant of claim 1 wherein the polylactic acid polyglycolic acid (PLGA) copolymer matrix comprises at least 40% of the weight of the drug implant.

8. (canceled)

9. A biodegradable drug implant of claim 1, wherein the drug implant is placed at or near the spine.

10. A biodegradable drug implant of claim 1, further comprising a clonidine compound, an analgesic, a GABA compound, a corticosteroid, a muscle relaxant, a non-steroidal anti-inflammatory compound, an anesthetic or a combination thereof.

11. A biodegradable drug implant of claim 10, wherein the drug implant comprises clonidine.

12. A biodegradable implant of claim 1, wherein the drug implant releases dexamethasone within 7 days after the implant is implanted under the skin.

13. A biodegradable implant of claim 1, wherein the PLGA copolymer matrix has a molecular weight of about 20,000 Da to 50,000 Da.

14. A biodegradable implant of claim 1, wherein the PLGA copolymer matrix has an inherent viscosity from about 0.10 dL/g to about 1.2 dL/g.

15. A biodegradable implant of claim 1, wherein the drug implant comprises pores that allows the release of the drug from the implant.

16. A method of treating radicular pain in a patient in need of such treatment, the method comprising administering a biodegradable drug implant to a target tissue site beneath the skin of the patient, the biodegradable drug implant comprising dexamethasone in an amount from about 50% to 80% of the weight of the drug implant, and a polylactic acid polyglycolic acid (PLGA) copolymer matrix, wherein the drug implant releases the dexamethasone over a period of at least about 30 days.

17. A method of treating radicular pain according to claim 16, wherein the radicular pain is caused by sciatica.

18. A method of treating radicular pain according to claim 16, wherein the biodegradable implant is administered at or near the spine.

19. A method of treating radicular pain according to claim 16, wherein biodegradable implant further comprises a clonidine compound, an analgesic, a GABA compound, a corticosteroid, a muscle relaxant, a non-steroidal anti-inflammatory compound, an anesthetic or a combination thereof.

20. (canceled)

21. A biodegradable implant of claim 1, wherein the PLGA copolymer matrix has an inherent viscosity from about 0.70 dL/g to about 0.90 dL/g.

22. A biodegradable implant of claim 1, wherein the PLGA copolymer matrix has an inherent viscosity from about 0.70 dL/g to about 0.90 dL/g and a modulus of elasticity from about 1×10² dyn/cm² to about 6×10⁵ dyn/cm².
